(19) United States
(12) Patent Application Publication
(10) Pub. No.: US 2005/0058980 A1
(43) Pub. Date: Mar. 17, 2005

(54) Germinant produced from IC-21 macrophages, method and use thereof

(75) Inventors: Bradford W. Gutting, Fredericksburg, VA (US); Tony L. Buhr, King George, VA (US); Lindsay A. Sobota, Spotsylvania, VA (US); Amanda S. Schilling, Fredericksburg, VA (US); Kelley S. Gaske, King George, VA (US)

Correspondence Address: Matthew J. Bussan, Esq., NSWCDD (XDC1), Dahlgren, VA, 22448-5100, US

(73) Assignee: Government of the United States of America as represented by the Secretary of the Navy

(21) Appl. No.: 10/666,826

(22) Filed: Sep. 16, 2003

(51) Int. Cl. A01N 1/02; C12N 1/12

(52) U.S. Cl. 435/2

(57) ABSTRACT

A spore germinant, particularly effective against endospores, is produced by IC-21 macrophages.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### BACKGROUND OF THE INVENTION

1. Field of the Invention

The present invention provides spore germinants produced by IC-21 macrophages.

2. Brief Description of the Related Art

Problematic for a number of putative germinants for Bacillus are inefficiency and special heat condition requirements. The first problem, inefficiency or lack of efficacy, exists as very few proposed germinants signal the synchronous germination of 1 log or more of bacteria, and there are none known that signal the synchronous germination of 2 logs or more of bacterial endospores. This is in contrast with most medical and decontamination protocols requiring at least 6-log reductions. The second problem is the requirement for a special heat condition. Most reported germinants are accompanied with a non-biological prerequisite in order to synchronize the germination, which is a heat activation at 65-70° C.

Previously published data from other investigators suggested that RAW264.7 macrophages secrete a germinant that may be useful against virulent endospore-forming bacteria (Sterne) but not against non-pathogenic strains (B. subtilis) (see Ireland, J. A. and P. C. Hanna, Infection and Immunity, 2002, 70: 5870-5872). The exact nature and identification of this suspected germinant has yet to be characterized.

There is a need in the art for a germinant that signals efficient germination under biological, non-heat shock conditions. The present invention addresses this and other needs.

### SUMMARY OF THE INVENTION

The present invention includes a spore germinant produced by IC-21 macrophages.

The present invention also includes a method for producing one or more germinants from IC-21 macrophages comprising the steps of providing IC-21 macrophages and adding spores to the provided IC-21 macrophages effective to produce germinant. The addition of spores may occur in a liquid medium, and filtering may be used to purify the product germinant.

Additionally, the present invention includes a method for germinating spores comprising the steps of providing one or more germinants from IC-21 macrophages and applying the germinants to spores effective to cause germination. Pathogenic and non-pathogenic bacteria spore germinants may be produced.

### BRIEF DESCRIPTION OF THE DRAWINGS

FIG. 1 shows a comparison of the colony forming units after B. globigii spores were treated for 30 minutes without IC-21 macrophage produced germinant (−Macs) or with IC-21 macrophage produced germinant (+Macs), before and after heat treatment;

FIG. 2 shows a comparison for the colony forming units for B. globigii spores treated without IC-21 macrophage produced germinant (−Macs), with IC-21 macrophage produced germinant (+Macs), and with phosphate buffered saline (PBS), before heat treatment; and, FIG. 3 shows a comparison for the colony forming units for B. globigii spores treated without IC-21 macrophage produced germinant (−Macs) and with IC-21 macrophage produced germinant (+Macs), before heat treatment and after heat treatment, for a time period of 5 minutes.

### DETAILED DESCRIPTION OF THE PREFERRED EMBODIMENT

The present invention includes a novel germinant, having one or more molecules, that can signal dormant bacterial endospores to germinate. The germinant is obtained from IC-21 macrophages, may be purified, and applied as a germinant to additional spores.

### Germinant Production

The germinant of the present invention is a bacterial spore germinant produced by IC-21 macrophages. IC-21 macrophages are provided in appropriate concentration, such as 1×10¹⁰ total in 0.5 ml growth media, 1×10⁶ total in 0.5 ml growth media, 1×10⁵ total in 0.5 ml growth media, and the like, with a preferred concentration of 1×10⁶ total in 0.5 ml growth media used. The IC-21 macrophages are preferably obtained from the American Type Culture Collection (ATCC) of Manassas, Va., under ATCC No. TIB-186 or other supplier. Once obtained the IC-2 1 macrophages are cultured in ATCC culture media (RPMI 1640) supplemented with 10% FBS (fetal bovine serum; supplied by Sigma Chemicals) and 5% Penicillin/Streptomycin (ATCC stock is 10,000 I.U./ml Penicillin and 10,000 μg/ml Streptomycin). Preferably, macrophages are seeded (adhered) in growth media (+FBS and Pen/Strep) for one hour and then all experimentation thereafter uses basal media (no FBS or antibiotics). Cells were cultured, passed and maintained using standard tissue culture techniques. Cells were passed no more than 30 times prior to experimentation. Prior to experimentation (see text), cells were washed in FBS and Penicillin/Streptomycin free media and experimentation was conducted in such a solution (i.e., basal media), and seeded. The growth media used for seeding IC-21 macrophages may include any appropriate growth media such as, for example without limitation, basal media, and other like media determinable by those skilled in the art. The IC-21 macrophages were allowed to adhere over a sufficient period of time, and washed. Washing may be repeated, as desired, generally using the seeding media composition.

The germinants are produced from IC-21 macrophages by adding spores to IC-21 macrophages in a manner effective to produce the germinants. When using spores to produce the germinant, spores are added to the IC-2 1 macrophages in effective amounts to produce germinant. Spore species include spore-forming bacterial species, with representative spores including, for example without limitation, Bacillus, and Clostridium, and the like. Effective amounts of spores may include one or more spores, components or simulated parts thereof, sufficient to induce the IC-macrophages to generate germinant. Preferably, the macrophages are exposed to spores in representative concentrations in an appropriate media, such as 1×10¹⁰ total in 0.5 ml media, 1×10⁶ total in 0.5 ml media, 1×10⁵ total in 0.5 ml media, and the like, with a preferred concentration of 1×10⁶ total in 0.5 ml media used. Appropriate media include for example without limitation, basal media, and other like media determinable by those skilled in the art. Preferred amounts of spores include a ratio amount of from about 100:1 to about 1:100, spores to macrophage, with more preferred amounts being a ratio amount of from about 10:1 to about 1:10, spores to macrophage, and a most preferred amount being a ratio amount of from about 1:1 to about 1:1, spores to macrophage. The spores may be processed from a spore/PBS (or spore/water) stock solution and prepared using standard microbiology techniques. In short, spores are grown to log-phase (approximately 10¹⁰CFU/ml) using appropriate growth media under optimal growth conditions (temperature and volume with shaking for aeration). After which, spores are purified using centrifugation, quantified, and stored in water at 4° C. until used.

Addition of the spores to the IC-21 macrophages is preferably done by wetting, or otherwise contacting, the washed IC-21 macrophages with the spore/media solution. Other methods may include shaking, mixing, stirring, other otherwise combining the IC-21 macrophages and spores with one or both components in solution. Once contacted, the spore/IC-21 macrophages combination is incubated. Incubation may include elevated temperatures such as, for example without limitation, from about 30° C. to about 50° C., with preferred temperatures from about 35° C. to about 40° C., such as about 37° C., with the temperature and other incubation conditions determinable by one skilled in the art.

When needed or desired, after the incubation of the spores and IC-21 macrophages, the product may be separated into supernatant, i.e., germinant, and solution, using known separation techniques such as, for example without limitation, filtering, centrifuge force, drying, column separation, and like, with filtering preferred. Filtering the germinant preferably includes a filter of from about 0.3 micrometers or less, with a filter of 0.2 micrometers most preferably used. In addition to filtering, the germinant product may be further purified or concentrated with such methods as centrifuging, drying, reduced pressure treatment, or other known methods of isolating the germinant from liquid or solid contaminants. Once dried, the germinant forms a solid mass that may be reconstituted with the addition of a solvent, such as alcohol, water, salt solutions, chemically reactive or chemically inert dyes, and/or bactericidal/bacteristatic agents, with water preferred.

### Germination Using Germinant

The supernatant, either in purified or non-purified form, is applied to a spore population in order to provide growth (and germination) thereto. Application of the germinant includes such methods as coating, shaking, flooding, washing, or otherwise contacting a surface or composition contaminated, or suspected to being contaminated, with the germinant in a manner to expose the spore population with the germinant. Once the germinant is applied, it is preferably maintained in contact with the spore population for an extended period of time, such as 15 minutes, one hour, one week and the like, with the period of time variable relative such factors as the amount of germinant available, the necessity to reuse a contaminated surface such as for example the necessity to use a carrier flight deck for combat flight operations, weather conditions, and other like factors determinable by those skilled in the art. Application of the germinants for germination include any appropriate amounts for germination such as, for example without limitation, germinant compositions having germinant present in an amount of from about 1×10¹² mg/L to about 1×10⁻¹² mg/L.

The spore germinant of the present invention may be effective across bacterial genuses and species. Germinants produced by adding one spore type may be applied to germinate spores of the same or different spore class, with selection of spores to produce germinant and spores to which apply the produced germinant determinable by those skilled in the art with routine experimentation. Preferably, the germinants of the present invention are produced by endospores of the Bacillus species, and most preferably the germinants includes Bacillus globigii endospores, with the germinant of the present invention preferably useful for germination of endospores, particularly endospores of the Bacillus species, such as Bacillus globigii and Bacillus anthracis. The germinant of the present invention may include germinants for both pathogenic and non-pathogenic bacteria strains.

### General Procedures

IC-21, RAW264.7 or J774A.1 macrophages (1×10⁶ total in 0.5 ml growth media) were seeded into one well of a 24 well tissue culture plate. Treatment Groups are identified as “+Macs” (+macrophages) in attached figures. All cell lines were obtained from ATCC and were passed <30 times prior to experimentation. The growth media used for seeding RAW264.7 consisted of basal media (RPMI1640, cat #11875-083 from Gibco, BRL) plus 1% Penicillin/Streptomycin (cat #30-2300, ATCC) and 10% fetal bovine serum (FBS, cat #30-2020, ATCC). The growth media used for seeding J774A. 1 consisted of basal media (DMEM, cat #30-2001 from ATCC) plus 1% Penicillin/Streptomycin (cat #30-2300, ATCC) and 10% fetal bovine serum (FBS, cat #30-2020, ATCC). The growth media used for seeding IC-21 included basal media (RPMI1640, cat #30-2001, ATCC) plus 1% Penicillin/Streptomycin (cat #30-2300, ATCC) and 10% fetal bovine serum (FBS, cat #30-2020, ATCC). Each cell line was allowed to adhere for 1 hour and then washed 2× with their respective basal media only (i.e., media that did not contain FBS or Pen/Strep). After washing, the macrophages were exposed to 1×10⁶ B. globigii spores (MOI=1) in 0.5 ml basal media. The spores were taken from a spore/PBS stock solution prepared using standard laboratory protocols. After 1 hour in an incubator (37° C., saturating humidly, 95% air/5% CO₂) the final volume in the well was adjusted to 1.2 ml with basal media, collected by pipet, and filtered through a 0.2 μm filter. With all cell lines, the control groups were wells that contained the respective media and B. globigii spores, but no macrophages.

Testing procedures to determine germinant potential were then used. The filter sterilized supernatant, i.e., free from macrophages and B. globigii spores, from both the +Macs and −Macs solutions was tested as a germinant on a fresh population of B. globigii spores. Testing included where 0.5 ml supernatant was added to a single well of a 24 well plate and then 1×10⁶ B. globigii spores were added. The plate was incubated for a specified time point (either 5 or 30 minutes) in a shaker incubator (200 RPM, 37° C.). After incubation, the spores were directly plated on LB agar (a measurement of spores+vegetative cells in the population) or heat-treated (65° C., 30 minutes) prior to plating (a measurement of spores only). Spore plating used standard serial dilution techniques.

As detailed herein, the germinant was produced from and tested on B. globigii spores; the IC-2 1 macrophages were exposed to B. globigii spores for a set time point and after this the supernatant (exposure media) was isolated, filtered and tested as the germinant on a fresh batch of B. globigii spores.

### EXAMPLE 1

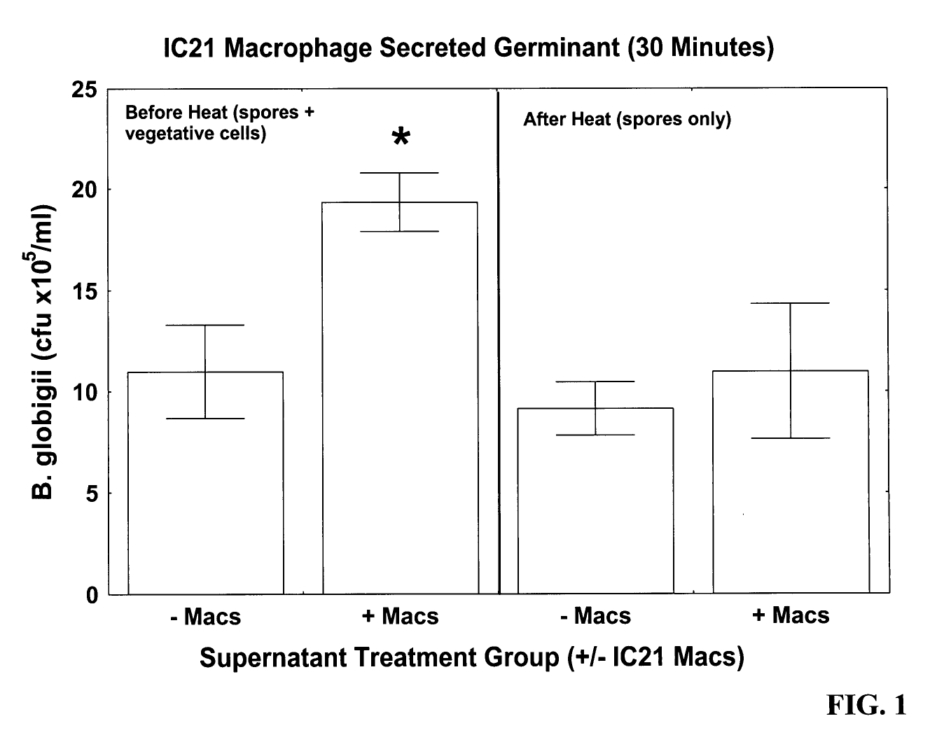

IC-21 macrophage produced germinant was produced and tested as a germinant. 1×10⁶ B. globigii spores were incubated for 1 hour at 37° C. under saturating humidity in an atmosphere of 5% CO₂ and 95% air with 1×10⁶ IC-21 macrophages (designated as “+Macs”) and separately with media alone (control group designated as “−Macs”). After the 1 hour incubation, the supernatant was filter sterilized with a 0.2 μm filter and tested as a germinant on a fresh preparation of spores. The +Mac supernatant and −Macs supernatant were added, in separate test batches, to 1×10⁶ B. globigii spores under incubation conditions for 30 minutes. After this incubation, colony forming units (CFU) were determined before heat treatment (listed as “Before Heat”) and after heat treatment (listed as “After Heat”), as shown in FIG. 1. Values indicate mean ±SEM (n=4 per group). Asterisks indicate significance (p<0.05) as determined using a one-way ANOVA with an LSD post-hoc comparison. The process was repeated with CFU determined before heat treatment, with the results shown in Table A.

In Table A, below, 1×10⁶ B. globigii spores were incubated for 1 hour (37° C., saturating humidity, 5% CO₂/95% air) with 1×10⁶ IC-21 macrophages (+Macs) or media alone (−Macs). After incubation, the supernatant was filter sterilized (0.2 μm) and tested as a germinant on a fresh preparation of spores. Here, +Mac supernatant or −Macs supernatant was added to 1×10⁶ B. globigii spores for 30 minutes. After incubation, CFU was determined before heat treatment. Values indicate mean ±SEM (n=4 per group). Asterisks indicate significance (p<0.05) as determined using Student's t test.

TABLE A

| | B. globigii |
|---|---|
| Treatment Group | (cfu × 10⁵/ml) |
| −Macs | 7.50 ± .29 |
| +Macs | 8.17 ± .06* |

### EXAMPLE 2

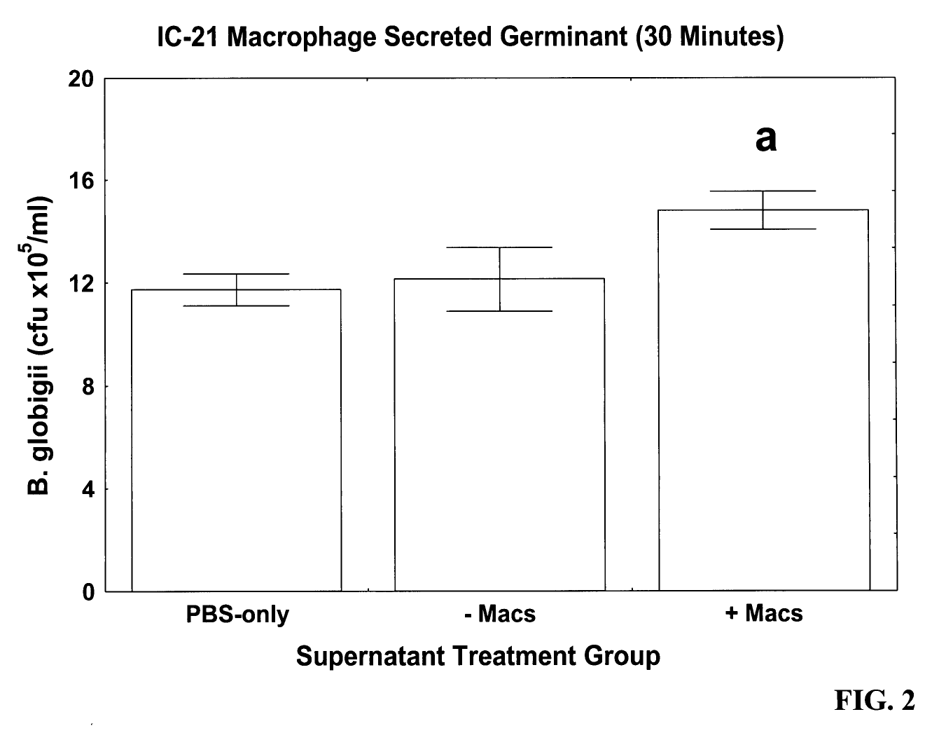

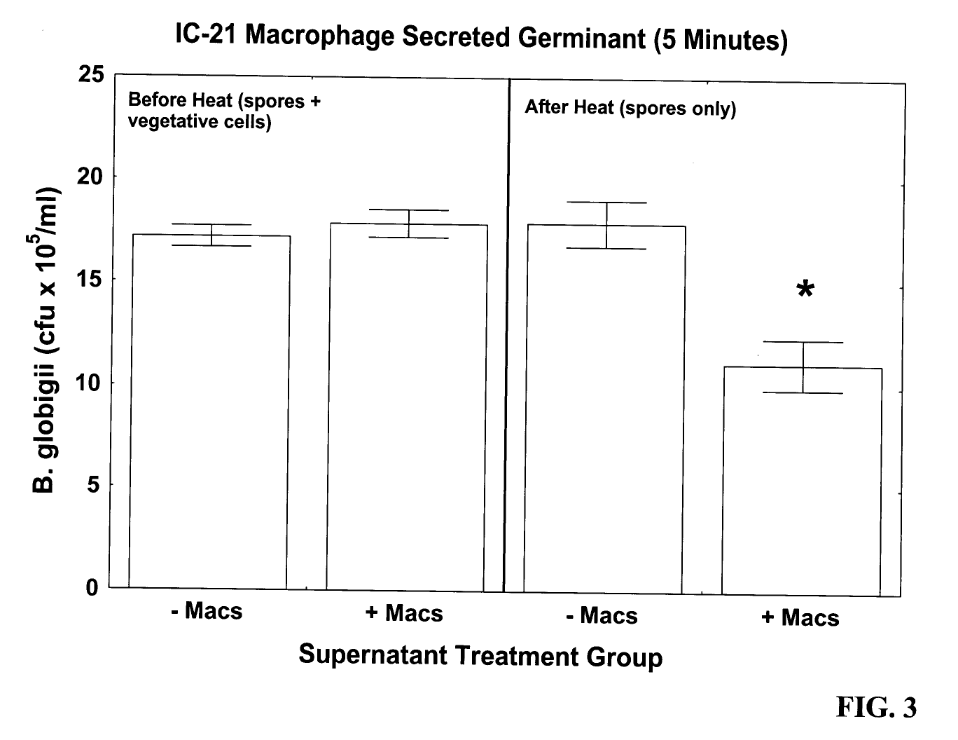

IC-21 macrophage produced germinant was produced and tested as a germinant. 1×10⁶ B. globigii spores were incubated for 1 hour at 37° C. under saturating humidity in an atmosphere of 5% CO₂ and 95% air with 1×10⁶ IC-21 macrophages (designated as “+Macs”) and separately with media alone (designated as “−Macs”). After the 1 hour incubation, the supernatant was filter sterilized with a 0.2 μm filter and tested as a germinant on a fresh preparation of spores. The +Mac supernatant and −Macs supernatant were added, in separate test batches, to 1×10⁶ B. globigii spores under incubation conditions for 30 minutes. For comparison, 1×10⁶ spores were also incubated with PBS for 30 minutes. After this incubation period, colony forming units (CFU) were determined before heat treatment, as shown in FIG. 2. Values indicate mean ±SEM (n=4 per group). ₐIndicates significance (p=0.06) as determined using a one-way ANOVA with an LSD post hoc comparison.

### EXAMPLE 3

IC-21 macrophage produced germinant was produced and tested as a germinant. 1×10⁶ B. globigii spores were incubated for 1 hour at 37° C. under saturating humidity in an atmosphere of 5% CO₂ and 95% air with 1×10⁶ IC-21 macrophages (designated as “+Macs”) and separately with media alone (designated as “−Macs”). After the 1 hour incubation, the supernatant was filter sterilized with a 0.2 μm filter and tested as a germinant on a fresh preparation of spores. The +Mac supernatant and −Macs supernatant were added, in separate test batches, to 1×10⁶ B. globigii spores under incubation conditions for 5 minutes. After this incubation, colony forming units (CFU) were determined before heat treatment (listed as “Before Heat”) and after heat treatment (listed as “After Heat”), as shown in FIG. 3. Values indicate mean ±SEM (n=4 per group). Asterisks indicate significance (p<0.05) as determined using a one-way ANOVA with an LSD post-hoc comparison.

As seen in Examples 1-3, germinant produced from IC-21 macrophages was shown as effective. The filtered sterilized supernatant obtained from IC-21 macrophages mixed with spores (treatment; +macs) or media plus spores-only (control; −macs) was added to 1×10⁶ fresh spores. After 30 minutes (Examples 1 and 2), the colony forming units (CFU) was determined before and after heat treatment. As shown in FIG. 1, the supernatant from media plus spores only had no effect on the CFU of spores after 30 minutes, regardless of heat treatment. However, the total number of spores roughly doubled when exposed to the IC-21 supernatant (before heat, left side of FIG. 1) and returned to base line after heat treatment. These results suggest that a small percent of the 1×10⁶ B. globigii spores germinated and replicated (vegetative heat sensitive cells) within 30 minutes. The increase in the total number of B. globigii cells following exposure to IC-21 macrophage supernatant was reproducible and observed in an independent experiment, as evidenced in Table A. The ability of supernatant from IC-21 macrophages to induce germination is more evident when compared to PBS-only (phosphate buffered saline) as exemplified in Example 2. In Example 2, spores were exposed to (i) PBS-only, (ii) IC-21 basal media only (−macs), or IC-21 macrophages for 30 minutes. In Example 3, the observations of spore growth were further defined, relative to Example 1, by changing the incubation time for supernatant mixed with spores from 30 minutes to 5 minutes. As shown in Example 3,and FIG. 3, there was no increase in total spores (spores plus vegetative cells; before heat) and the number of CFU decreased following heat treatment (spores only; after heat).

### Comparative Data

As shown in Examples 4-6 (and Tables 1-3), below, there was no detection of a significant germinant effect using supernatant from RAW264.7 or J77A4.1 macrophages. There was an indication that the culture media used in RAW264.7 macrophages was able to induce germination in one experiment (see Table 1), but this was not reproducible.

### EXAMPLE 4 (Comparative Data)

RAW264.7 and J77A4.1 macrophages were used to produce supernatant to be tested as a germinant 1×10⁶ B. globigii spores were incubated for 1 hour at 37° C. under saturating humidity in an atmosphere of 5% CO₂ and 95% air with 1 x 106 RAW264.7 and J77A4.1 macrophages (designated as “+Macs”) and separately with media alone (designated as “−Macs”). After the 1 hour incubation, the supernatant was filter sterilized with a 0.2 μm filter and tested as a germinant on a fresh preparation of spores. The +Mac supernatant and −Macs supernatant were added, in separate test batches, to 1×10⁶ B. globigii spores under incubation conditions for 30 minutes. After this incubation, colony forming units (CFU) were determined before heat treatment (listed as “No Heat”) and after heat treatment (listed as “Plus Heat”), as shown below in Table 1. Values indicate mean ±SEM (n=4 per group).

TABLE 1

B. globigii
(CFU × 10⁵/ml)

| Supernatant Treatment | | No Heat | Plus Heat |
|---|---|---|---|
| RAW264.7 Cells | −Macs | 20.6 ± 2.4 | 9.8 ± 1.7 |
| | +Macs | 26.3 ± 1.8 | 10.7 ± 4.3 |
| J774A.1 Cells | −Macs | 15.7 ± 3.0 | 8.7 ± 1.0 |
| | +Macs | 13.2 ± 2.7 | 8.5 ± 2.0 |

### EXAMPLE 5 (Comparative Data)

RAW264.7 and J77A4.1 macrophages were used to produce supernatant to be tested as a germinant. 1×10⁶ B. globigii spores were incubated for 1 hour at 37° C. under saturating humidity in an atmosphere of 5% CO₂ and 95% air with 1×10⁶ RAW264.7 and J77A4.1 macrophages (designated as “+Macs”) and separately with media alone (designated as “−Macs”). After the 1 hour incubation, the supernatant was filter sterilized with a 0.2 μm filter and tested as a germinant on a fresh preparation of spores. The +Mac supernatant and −Macs supernatant were added, in separate test batches, to 1×10⁶ B. globigii spores under incubation conditions for 30 minutes. For comparison, 1×10⁶ spores were also incubated with PBS for 30 minutes. After this incubation, colony forming units (CFU) were determined before heat treatment (listed as “No Heat”) and after heat treatment (listed as “Plus Heat”), as shown below in Table 2. Values indicate mean ±SEM (n=4 per group).

TABLE 2

B. globigii
(CFU × 10⁵/ml)

| Supernatant Treatment | | No Heat | Plus Heat |
|---|---|---|---|
| RAW264.7 Cells | −Macs | 7.53 ± 3.06 | Not Tested |
| | +Macs | 7.10 ± 16.5 | Not Tested |
| J774A.1 Cells | −Macs | 6.80 ± 8.54 | Not Tested |
| | +Macs | 6.30 ± 1.73 | Not Tested |
| PBS - only | | 6.80 ± 11.3 | Not Tested |

The process was repeated with CFU determined before heat treatment, with the results shown below in Table 3.

TABLE 3

B. globigii
(CFU × 10⁵/ml)

| Supernatant Treatment | | No Heat | Plus Heat |
|---|---|---|---|
| RAW264.7 Cells | −Macs | 11.9 ± 2.51 | Not Tested |
| | +Macs | 12.4 ± 1.45 | Not Tested |
| J774A.1 Cells | −Macs | 11.7 ± 12.9 | Not Tested |
| | +Macs | 9.4 ± 16.7 | Not Tested |
| PBS - only | | 11.7 ± 6.12 | Not Tested |

The spore germinant product of the present invention is useful in germinating dormant spores at a given time, particularly contaminant spores that are known hazards. These hazardous spores are germinated by applying the produced IC-21 macrophages germinants to the problematic spores in a manner effective to cause germination of the problematic spores. Germination includes all cellular processes, morphological, genetic and physiological changes, which occur as a bacterial endospore breaks dormancy and transitions into a metabolically active, vegetative bacterium, with gemination including loss of the spore coat and uptake of water.

The present invention provides a safe and effective spore germinant. As the germinant was isolated from mammalian cells, its use is generally safe, i.e., non-toxic. Additionally, the germinant shows effectiveness without the need to concentrate the sample for germinant effect. The germinant of the present invention appears distinct and non-present from germinant from RAW264.7 macrophages, while working to germinate non-pathogenic B. globigii spores.

The germinant of the present invention has a wide number of potential applications, such as use in bacterial decontamination solutions (by coupling with bactericidal agents for example) and strategies including, without limitation, decontamination of endospore-forming bacteria for the military, medical industry, household areas and in the event of terrorist attacks. Other applications includes use in human or veterinary medications that are used either externally or internally to control endospore-forming pathogens, industrial microbiology and agriculture. Endospore forming bacteria are used in industrial microbiology to produce biological compounds such as enzymes. The germinant described herein would be highly useful to control population growth in order to control production and costs. In agriculture, endospore-forming bacteria have well-documented uses, for example, Bacillus thuringiensis is used in both bacterial form and to produce the Bt toxin, which are used in insect biocontrol and as an insecticide, respectively. The germinant of the present invention may have roles for production of these bacteria and the associated toxin.

The foregoing summary, description, and examples of the present invention are not intended to be limiting, but are only exemplary of the inventive features which are defined in the claims.

1. A spore germinant produced by IC-21 macrophages.

2. A method for producing one or more germinants from IC-21 macrophages, comprising the steps of:
providing IC-21 macrophages;
adding spores to the provided IC-21 macrophages effective to produce germinant.

3. The method of claim 2, wherein the added spores to the IC-21 macrophages occurs in solution.

4. The method of claim 3, further comprising the step of filtering the produced germinant from solution.

5. The method of claim 2, wherein the step of adding spores occurs in a ratio amount of from about 100:1 to about 1:100, spores to macrophage.

6. The method of claim 2, wherein the step of adding spores occurs in a ratio amount of from about 10:1 to about 1:10, spores to macrophage.

7. The method of claim 6, wherein the step of adding spores occurs in a ratio amount of from about 1:1 to about 1:1, spores to macrophage.

8. The method of claim 2, wherein the step of filtering comprises a filter of from about 0.2 micrometers or less.

9. The method of claim 4, further comprising the step of concentrating the filtered germinant.

10. The spore germinant product produced by the process of claim 2.

11. A method for germinating spores, comprising the steps of:
providing one or more germinants from IC-21 macrophages; and,
applying the germinants to spores effective to cause germination.

12. The method of claim 11, wherein the step of providing one or more germinants comprises endospores of the Bacillus species.

13. The method of claim 12, wherein the step of providing one or more germinants comprises endospores of the Bacillus globigii.

14. The method of claim 11, wherein the step of applying the germinants comprises application to spores of the same spore class as used to produce germinant.

15. The method of claim 11, wherein the step of applying the germinants comprises application to spores of a different spore class as used to produce germinant.

16. The method of claim 11, wherein the spores comprise endospores.

17. The method of claim 16, wherein the endospores comprise Bacillus species.

18. The method of claim 17, wherein the endospores comprise Bacillus globigii.

19. The method of claim 11, wherein the step of applying the germinants comprises a germinant composition with germinant present in an amount of from about 1×10¹² mg/L to about 1×10⁻¹² mg/L.

20. A non-pathogenic bacteria strain spore germinant.
